SEQUENCE LISTING (1 sequence)

SEQ ID NO: 1 - protein, 13 aa, Artificial
  synthetic peptide amino acid sequence
VHFFKNIVTPRTP

(19) United States
(12) Patent Application Publication
(10) Pub. No.: US 2013/0210054 A1
(43) Pub. Date: Aug. 15, 2013

(54) Amino Acid Copolymer Assay

(71) Applicant: Momenta Pharmaceuticals, Inc., (US)

(72) Inventor: Josephine S. D'Alessandro, Marblehead, MA (US)

(73) Assignee: Momenta Pharmaceuticals, Inc., Cambridge, MA (US)

(21) Appl. No.: 13/680,452

(22) Filed: Nov. 19, 2012

Related U.S. Application Data
(60) Provisional application No. 61/597,051, filed on Feb. 9, 2012.
(60) Provisional application No. 61/614,200, filed on Mar. 22, 2012.

(51) Int. Cl. C12Q 1/02

(52) U.S. Cl. CPC C12Q 1/025
USPC 435/29

(57) ABSTRACT

The present disclosure relates to polarized type 2 T helper (Th2) cells and methods for using such cells in the assessment of antigenic compositions.

### RELATED APPLICATIONS

The present application claims the benefit of U.S. Provisional Patent Application Ser. Nos. 61/597,051, filed Feb. 9, 2012, and 61/614,200, filed Mar. 22, 2012, both of which are herein incorporated by reference in their entireties.

### REFERENCE TO SEQUENCE LISTING

Pursuant to 37 C.F.R. 1.821(c), a sequence listing is submitted herewith via EFS-Web as an ASCII compliant text file named “Sequencelisting.txt” that was created on Nov. 14, 2012, and has a size of 542 bytes. The content of the aforementioned file named “Sequencelisting.txt” is hereby incorporated by reference in its entirety.

### TECHNICAL FIELD

The present disclosure relates to polarized type 2 T helper (Th2) cells and methods for using such cells in the assessment of antigenic compositions.

### BACKGROUND

Glatiramer acetate (marketed as the active ingredient in COPAXONE® by Teva Pharmaceutical Industries Ltd., Israel) is used in the treatment of the relapsing-remitting form of multiple sclerosis (RRMS). According to the COPAXONE® product label, glatiramer acetate (GA) consists of the acetate salts of synthetic polypeptides, containing four naturally occurring amino acids: L-glutamic acid, L-alanine, L-tyrosine, and L-lysine with a reported average molar fraction of 0.141, 0.427, 0.095, and 0.338, respectively. Chemically, GA is designated L-glutamic acid polymer with L-alanine, L-lysine and L-tyrosine, acetate (salt). Its structural formula is:

(Glu,Ala,Lys,Tyr)ₓ.xCH₃COOH (C₅H₉NO₄.C₃H₇NO₂.C₆H₁₄N₂O₂.C₉H₁₁NO₃)ₓ.xC₂H₄O₂ CAS-147245-92-9

Multiple sclerosis (MS) is an autoimmune disease of the central nervous system (CNS) believed to be caused by a breakdown in immune tolerance related to a proinflammatory Th1/Th17 imbalance. Upon migration through the blood brain barrier, these pathogenic, autoreactive Th1/Th17 cells and their associated cytokines contribute to the destruction of the myelin sheath surrounding neuronal axons.

One of the key biological effects of GA is thought to be the induction of a Th2 cell response producing tolerogenic cytokines Induction of Th2 cytokine response has been identified in human MS patients administered GA therapy. Further, GA-reactive Th2 cells can confer protection in the CNS against experimental autoimmune encephalomyelitis (EAE) (murine model of MS) in recipient mice by a process termed “bystander suppression.”

### SUMMARY OF THE INVENTION

The invention is based, at least in part, on the development of Th2 polarized T cells such as the glatiramer acetate (GA)-specific type 2 T helper (Th2) cells disclosed herein, e.g., (GA)-specific Th2 cells that are capable of expressing high levels of one or more (e.g., 2, 3, or 4, 5 or fewer than 5, 6 or fewer than 6, 7 or fewer than 7, and so on, including all cytokines or markers disclosed herein or a subset thereof) Th2-specific cytokines (e.g., interleukin (IL-4), IL-5, IL-10, and IL-13) when exposed to GA in the presence of antigen-presenting cells (APC). Thus, the GA-specific Th2 cells express various Th2-specific cytokines in response to restimulation with antigen (GA) presented by APCs in vitro. The GA-specific Th2 cells described herein have the surprising and unexpected characteristic of being able to reproducibly distinguish GA from certain other non-conforming amino acid copolymers and/or to confirm that GA, or a sample, lot, or batch thereof, conforms to a reference value for GA. In particular, the GA-specific Th2 cells described herein express reproducibly and significantly decreased levels of Th2 cytokines (e.g., IL-4, IL-5, IL-10, and IL-13), as well as of IL-2, IL-6 and TNF-α, when exposed to (restimulated in the presence of) APCs and non-conforming amino acid copolymers (e.g., COP-1 (Poly(Ala, Glu, Lys, Tyr) 6:2:5:1 hydrobromide (mw 20,000-30,000; Sigma-Aldrich, St. Louis, Mo.), and non-conforming-GA (see Example 5)), compared to when restimulated in the presence of APCs and GA.

Thus, as used herein, the term “GA-specific type 2 T helper (Th2) cells” refers to a polyclonal population of Th2 cells that are specific for GA and have less than 1% cross-reactivity with MBP, and express more (e.g., at least about 1.2, 1.3, 1.4, or 1.5 fold more) interleukin IL-4 when incubated for 24 hours in the presence of 25 μg/ml GA and antigen-presenting cells (APCs) than when incubated in the presence of the same concentration of poly(Ala, Glu, Lys, Tyr) 6:2:5:1 hydrobromide having a molecular weight of 20,000-30,000 Daltons and APCs. The term “polyclonal population of Th2 cells” refers to a population of Th2 cells, or subpopulations of the Th2 cells selected or isolated from the polyclonal population of Th2 cells, having T cell receptors that recognizes the same antigen (GA), but not necessarily the same GA peptide/MHC molecule complex. “Percent (%) cross-reactivity”, as used herein, is determined by dividing the amount of a GA-induced polypeptide (e.g., IL-4, IL-5, IL-6, IL-13) that is produced by a GA-specific Th2 cell cultured in the presence of APCs and MBP by the amount of the same GA-induced polypeptide that is produced by a GA-specific Th2 cell cultured in the presence of APCs and GA, and multiplying that number by 100%. In a specific instance, the GA-induced polypeptide is IL-4. In some instances, the MBP that can be used to determine cross-reactivity is MBP (87-99), having amino acid sequence available, e.g., from Peptides International, Inc. (Louisville, Ky.) (Catalog No. PMB-3973-PI). In other instances the MBP that can be used to determine cross-reactivity is murine MBP, e.g., available from Enzo Life Sciences, Inc. (Farmingdale, N.Y.) (Catalog No. ALX-202-075). In certain instances, the MBP (e.g., murine MBP) that can be used to determine cross-reactivity is obtained according to the method described in U.S. Pat. No. 5,849,886 to Määtä, and in Määtä, et al. Biochem Biophys Res Commun. 1997 238:498. In some instances, if the level of the GA-induced polypeptide (e.g., IL-4, IL-5, IL-6, IL-13) is below the limit of detection of a test described herein (e.g., ELISA or multiplex assay), for purposes of determining % cross-reactivity, the lower limit of detection of the assay used to measure the level of the GA-induced polypeptide can be used (instead of 0). In certain instances, GA-specific Th2 cells have less than 5%, 4%, 3%, 2%, 1%, or less than 0.5% cross-reactivity with MBP. In certain instances, GA-specific Th2 cells are said to have “undetectable cross-reactivity” if the GA-specific Th2 cells do not produce detectable levels of at least one GA-induced polypeptide, as disclosed herein.

In some instances, compositions and methods disclosed herein can be used in assays that are relevant to a biological activity of GA. Such assays are desirable because they are useful in predicting the clinical function of GA and compositions comprising GA. Thus, the assays disclosed herein can be used in the validation and/or commercial release of GA. For example, the GA-specific Th2 cells described herein exhibit in vitro a therapeutic function of Th2 cells thought to be induced by GA, but not by certain other copolymers and proteins (e.g., one or more of COP-1 and/or myelin basic protein (MBP)) in vivo, and therefore provide a novel in vitro tool useful in predicting the in vivo therapeutic efficacy of an amino acid copolymer preparation (e.g., GA). The GA-specific Th2 cells described herein are also useful because they can express a high level of one or more Th2 cytokines when restimulated by GA and APCs, thereby facilitating accurate measurement of the one or more cytokines.

Described herein are compositions that comprise GA-specific Th2 cells which express more IL-4, and preferably at least 10-fold more IL-4, when incubated in the presence of a given concentration of GA and APCs than when incubated in the presence of the same concentration of MBP and APCs. In certain instances, the GA-specific Th2 cells, which are non-MBP reactive Th2 cells, express at least 100-fold (e.g., 100-fold, 1.000-fold, 10.000-fold) more IL-4 when incubated in the presence of a given concentration of GA and APCs than when incubated in the presence of the same concentration (e.g., 20 μg/ml, 40 μg/ml) of MBP and APCs. In some cases there is no dose-dependent increase in IL-4 expression when the GA-specific Th2 cells are exposed to MBP and APCs. In certain aspects, the predetermined range of IL-4 is 3000-5000 pg/ml when the GA-specific Th2 cells are incubated at a concentration of 1×10⁶ Th2 cells/ml for at least about 24 hours in the presence of the APCs and at least about 25 μg/ml of the sample.

Also described is a method comprising incubating at least one sample of a batch of a composition comprising glatiramer acetate (GA) in the presence of GA-specific Th2 cells and APCs, wherein the GA-specific Th2 cells have less than 1% cross-reactivity with myelin basic protein (MBP); determining the level of at least one GA-induced polypeptide (e.g., IL-4, IL-5, IL-6, and/or IL-13) expressed by the GA-specific Th2 cells at a predetermined time point; and formulating at least a portion of the batch as a drug product if the level of the at least one GA-induced polypeptide is within a predetermined range. In some instances, the GA-specific Th2 cells have a cross-reactivity with MBP of less than 0.5%. In some instances, the GA-specific Th2 cells, when incubated at a concentration of 1×10⁶ Th2 cells/ml in the presence of APCs and at least 15 μg/ml GA, produce at least 100-fold more IL-4 than when incubated in the presence of APCs and a control antigen. As used herein, a “control antigen” can be “no antigen” (e.g., only PBS or media), or an antigen that is known not to cross-react with the GA-specific Th2 cells (e.g., OVA). In some instances, the predetermined range is a specification for commercial release of GA. In other instances, the predetermined range is 125% to 65%, 125% to 70%, 125% to 75%, 120% to 70%, 115% to 70%, 110% to 70%, 105% to 70%, 100% to 70%, or 90 to 110% of the level of the same GA-induced polypeptide expressed by the GA-specific Th2 cells when incubated under the same conditions with a reference sample. In some instances, the reference sample is a sample of a commercially available pharmaceutical preparation of GA (e.g., Copaxone®).

In certain instances, the GA-specific Th2 cells express about 1.5 fold more interleukin IL-4 when incubated for at least 24 hours in the presence of at least 25 μg/ml GA and antigen-presenting cells (APCs) than when incubated in the presence of the same concentration of poly(Ala, Glu, Lys, Tyr) 6:2:5:1 hydrobromide having a molecular weight of 20,000-30,000 Daltons and APCs.

In certain instances, the GA-specific Th2 cells are a population of isolated polyclonal GA-specific Th2 cells. As used herein, a “population of isolated polyclonal GA-specific Th2 cells” consists of at least 70%, 75%, 80%, 85%, 90%, 95%, 96%, 97%, 98%, 99% or greater GA-specific Th2 cells relative to all other cells in the population.

In other instances, the GA-specific Th2 cells are non-human cells. The GA-specific Th2 cells can also be murine cells. In some instances, the GA-specific Th2 cells are not from a patient diagnosed with multiple sclerosis.

In some aspects, the pre-determined time point is at least 24 hours after starting the incubation of the composition comprising GA in the presence of the GA-specific Th2 cells and the APCs. In some instances, the pre-determined time point can be at least 48 hours after starting the incubation of the composition comprising GA in the presence of the GA-specific Th2 cells and the APCs. In certain instances, the GA-specific Th2 cells are incubated with the APCs at a ratio in the range of 1:3 to 1:10 (Th2 cells:APCs). In one instance, the GA-specific Th2 cells are incubated with the APCs at a ratio of 1:3 (Th2 cells:APSc). In some aspects, the formulating step comprises adding mannitol to the portion of the batch. In other aspects, the batch of a composition comprising GA is a drug substance.

In certain instances, the batch of the composition comprising glatiramer acetate is prepared by a method comprising: polymerizing N-carboxy anhydrides of L-alanine, benzyl-protected L-glutamic acid, trifluoroacetic acid (TFA)-protected L-lysine, and L-tyrosine to generate a protected copolymer (Intermediate-1); treating the protected copolymer to partially depolymerize the protected copolymer and to deprotect benzyl protected groups thereby producing Intermediate-2; treating Intermediate-2 to deprotect TFA-protected lysines thereby producing Intermediate-3; and processing Intermediate-3 to produce GA. Also described is a method of identifying a batch of a composition comprising a copolymer of tyrosine, lysine, alanine and glutamic acid as a batch of a composition comprising GA, the method comprising: providing a batch of a composition comprising a copolymer of tyrosine, lysine, alanine and glutamic acid; incubating at least one sample of the batch) in the presence of GA-specific type 2 T helper (Th2) cells and antigen-presenting cells (APCs), wherein the GA-specific Th2 cells have less than 1% cross-reactivity with myelin basic protein (MBP); determining the level of at least one GA-induced polypeptide expressed by the GA-specific Th2 cells at a predetermined time point; and identifying the batch of the copolymer as GA if the level of the at least one GA-induced polypeptide is within a predetermined range.

Also described is a method of selecting a batch of a composition comprising an antigen composition (such as an amino acid copolymer) with a pre-defined/selected signature or characteristics (e.g., GA), the method comprising: incubating at least one sample of the batch of the composition in the presence of GA-specific Th2 cells and APCs, wherein the GA-specific Th2 cells express more IL-4, and preferably at least 10-fold more IL-4, when incubated in the presence of a given concentration of GA and APCs than when incubated in the presence of the same concentration of a non-conforming amino acid copolymer (e.g., one or more of MBP and/or COP-1) and APCs under the same conditions; determining the level of at least one GA-induced polypeptide expressed by the GA-specific Th2 cells at a predetermined time point; and selecting the batch, as GA, if the level of the at least one GA-induced polypeptide is within a predetermined range. Where the level of the at least one GA-induced polypeptide is within a predetermined range or has a preselected relationship with a reference value, the method can include: providing and/or receiving information regarding the predetermined range or preselected relationship to another party (e.g., a party manufacturing GA), classifying, selecting, accepting, discarding, releasing, or withholding a batch of GA; reprocessing a batch through a previous manufacturing step; processing a batch of GA into drug product, shipping the product from a batch of GA, moving the batch of GA to a new location; or formulating, labeling, packaging, selling, offering for sell, releasing a batch of GA into commerce and/or directing any of the above actions. In some instances, the at least one GA-induced polypeptide includes IL-4.

Also described is a method of preparing a pharmaceutical composition comprising an amino acid copolymer, the method comprising: incubating at least one sample of a batch of a composition comprising an amino acid copolymer in the presence of GA-specific Th2 cells and APCs, wherein the GA-specific Th2 cells express more IL-4, preferably at least 10-fold more IL-4, when incubated in the presence of a given concentration of GA and APCs than when incubated in the presence of the same concentration of a non-conforming amino acid copolymer (e.g., one or more of COP-1 and/or myelin basic protein (MBP)) and APCs (e.g., naïve APCs); determining the level of at least one GA-induced polypeptide expressed by the GA-specific Th2 cell at a predetermined time point; selecting the batch, as GA, if the level of the at least one GA-induced polypeptide is within a predetermined range; and preparing a pharmaceutical composition comprising at least a portion of the selected batch. In some instances, the at least one GA-induced polypeptide includes IL-4.

Also described is a composition comprising GA-specific Th2 cells, prepared by the following method: culturing in vitro CD4+ T cells (e.g., enriched by negative selection), obtained from a subject (or subjects) (e.g., a mouse or mice) or cell line (e.g., an in vitro cell line) previously immunized with or exposed to GA (e.g., at least about 11 days prior to obtaining the cells, e.g., immunized with complete Freund's adjuvant), in the presence of GA and antigen-presenting cells (APCs) (e.g., at a ratio of APCs to T cells of 10:1 or 5:1) in culture media for about 2 to 4 days (e.g., 4 days or about 96 hours); exposing the cultured CD4+ T cells to GA (e.g., about 20 μg/ml) and APCs (i.e., restimulating in the presence of GA and APCs), wherein, before the exposure (restimulation), the cultured CD4+ T cells are propagated for at least about 10 days (e.g., in the presence of IL-2). The GA-specific Th2 cells can express one or more of the cytokines IL-2, IL-4, IL-5, IL-13, IL-6, IL-10, and tumor necrosis factor (TNF)-alpha when restimulated with GA in the presence of APCs. The CD4+ T cells can be isolated, e.g., from a subject's or subjects' draining lymph nodes. The APCs may be, but not necessarily, splenic, syngeneic to the CD4+ T cell, freshly isolated, naïve, and/or previously treated with an anti-proliferative agent. Preferably ratios of APCs to CD4+ T cells are about 5:1 or 10:1. The CD4+ T cells can be restimulated 1 or more, 2 or more, 3 or more, 4 or more, 5 or more, 6 or more, 7 or more, 8 or more, etc. times prior to use and/or storage. The CD4+ T cells can be further expanded in the presence of interleukin (IL)-2. Preferably, the GA-specific Th2 cells express more interleukin (IL)-4 when incubated in the presence of a given concentration of GA and antigen-presenting cells (APCs) than when incubated in the presence of the same concentration of a non-conforming amino acid copolymer (e.g., myelin basic protein (MBP) and/or COP-1) and APCs.

Also described are compositions comprising GA-specific Th2 cells, prepared by the method exemplified in FIG. 6.

Also described herein is a method for preparing a pharmaceutical composition comprising GA, comprising: polymerizing N-carboxy anhydrides of L-alanine, benzyl-protected L-glutamic acid, trifluoroacetic acid (TFA) protected L-lysine and L-tyrosine to generate a protected copolymer; treating the protected copolymer to partially depolymerize the protected copolymer and deprotect benzyl protected groups and deprotecting TFA-protected lysines to generate GA; and purifying the GA, wherein the improvement comprises: measuring the amount of at least one GA-induced polypeptide expressed by one or more GA-specific Th2 cells exposed to the GA in the presence of antigen-presenting cells (APCs). In some instances, the improvement further comprises: selecting the purified GA for use in the preparation of a pharmaceutical composition when the expression of the at least one GA-induced polypeptide is within a predetermined range. In other instances, the improvement can further comprise preparing a pharmaceutical composition comprising at least a portion of the selected purified GA. In some instances, the step of measuring the amount of at least one GA-induced polypeptide comprises determining the level of at least one GA-induced polypeptide expressed by the GA-specific Th2 cells at a predetermined time point; and selecting the GA if the level of the at least one GA-induced polypeptide is within a predetermined range. In certain instances, the GA-specific Th2 cells express more interleukin (IL)-4 when incubated in the presence of a given concentration of GA and APCs than when incubated in the presence of the same concentration of a non-conforming amino acid copolymer (e.g., MBP and/or COP-1) and APCs. In some instances, the at least one GA-induced polypeptide includes IL-4.

As used herein, a “copolymer”, “amino acid copolymer” or “amino acid copolymer preparation” is a heterogeneous mixture of polypeptides comprising a defined plurality of different amino acids (typically between 2-10, e.g., between 3-6, different amino acids). A copolymer may be prepared from the polymerization of individual amino acids. The term “amino acid” is not limited to naturally occurring amino acids, but can include amino acid derivatives and/or amino acid analogs. For example, in an amino acid copolymer comprising tyrosine amino acids, one or more of the amino acids can be a homotyrosine. Further, an amino acid copolymer having one or more non-peptide or peptidomimetic bonds between two adjacent residues is included within this definition. A copolymer is non-uniform with respect to the molecular weight of each species of polypeptide within the mixture.

As used herein, a “non-conforming amino acid copolymer” or a “non-conforming-GA” is a negative control material distinguishable from GA, or a reference value thereof, by an assay disclosed herein. For example, one which does not have, e.g., the same ratio of L-glutamic acid, L-alanine, L-tyrosine, and L-lysine as GA, and/or is otherwise distinguishable from GA in molecular weight, molecular weight distribution, pyro-glutamate content, the presence of contaminating agents (e.g., agents not present in GA or that are present in GA but at controlled or regulated levels) etc., and thus, is distinguishable from GA. In some cases a non-conforming amino acid copolymer has one or more of the following characteristics: a molecular weight outside of 6,500-7,500 Da; a molecular weight outside of 5,000-9,000 Da; a pyro-glutamate content outside of 2,000-7,000 ppm; a pyro-glutamate content outside of 2,500-5,500 ppm; a pyro-glutamate content outside of 3,000-5,000 ppm; a pyro-glutamate content outside of 3,500-4,500 ppm; a pyro-glutamate content outside of 5,000-9,000 ppm; a pyro-glutamate content outside of 6,500-7,500 ppm; and molar ratio of L-glutamic acid:L-alanine: L-tyrosine: L-lysine outside of: 0.141:0.427:0.095:0.338.

As used herein, the term “expressed” in the context of a polypeptide expressed by a Th2 cell means the Th2 cell has detectable levels of mRNA encoding the polypeptide. The mRNA may or may not be translated into the encoded polypeptide; though preferably the encoded polypeptide is translated and, preferably, although not necessarily, the polypeptide is secreted by the Th2 cell. The expression of a polypeptide can be determined, e.g., by quantifying the levels of mRNA encoding the polypeptide, e.g., by quantitative reverse transcriptase polymerase chain reaction (qRT-PCR), and/or by quantifying the intracellular levels of the polypeptide (e.g., by intracellular immunostaining), and, if the polypeptide is secreted, by quantifying the levels of the polypeptide in the cell culture media (e.g., by ELISA or multiplex immunoassay). As used herein, a “plurality of polypeptides” means two or more polypeptides.

As used herein, a “reference value” is a range or level of at least one GA-induced polypeptide (e.g., at least one GA-induced polypeptide disclosed herein) expressed by the GA-specific Th2 cells described herein in the presence of a concentration of GA. In some instances, the at least one GA-induced polypeptide includes IL-4. In some instances, the GA is the active ingredient in COPAXONE®. In some instances, a reference value is a specification for commercial release of a drug product comprising GA. For example, the specification for commercial release can be the specification required by the U.S. Food & Drug Administration (FDA), the European Medicines Agency (EMA), or the U.S. Pharmacopeial Convention (USP), e.g., for the pharmaceutical release of GA, or as provided on the label of a product approved by the FDA, the EMA, or the USP.

In some instances, the level of at least one GA-induced polypeptide is within a “predetermined range” when the level of the at least one GA-induced polypeptide is within a reference value range or when the level or the at least one GA-induced polypeptide is within about 5%, 10%, 20%, 30%, 40%, or 50% of a reference value level, e.g., under suitable conditions. In some instances, suitable conditions include use of the same or comparable concentrations of GA (e.g., in the determination of the reference value and the measured level) and/or use of the same of comparable detection techniques, as described herein. As used herein, “determining”, e.g., in the Th2 amino acid copolymer assay described herein, includes for example, monitoring, assaying, measuring, analyzing, detecting, reviewing, evaluating, correlating and/or estimating.

In some instances, the predetermined range is a specification for commercial release of GA. As disclosed herein, the specification for commercial release of GA may be determined by measuring, obtaining, assaying, etc., the level(s) of one or more GA-induced polypeptides (e.g., IL-4, IL-5, IL-6, IL-10, IL-13) produced by the GA-specific Th2 cells disclosed herein when cultured in the presence of APCs and a commercial pharmaceutical preparation of GA (e.g., Copaxone®). The specification for commercial release is typically within a percent range of the value determined, e.g., within 125% to 65%, 125% to 70%, 125% to 75%, 120% to 70%, 115% to 70%, 110% to 70%, 105% to 70%, 100% to 70%, or 90% to 110%, of the determined value. A non-limiting, exemplary specification for commercial release, can be, for example, 125% to 75% of the amount of IL-4 measured in the Th2 assay described herein when the GA-specific Th2 cells are cultured in the presence of APCs and 50 μg/ml of a commercial preparation of GA (Copaxone). As shown in FIG. 4, the amount of IL-4 measured under these conditions is about 4 μg/ml. Thus, a specification for commercial release can be about 3 μg/ml to 5 μg/ml IL-4.

While the present disclosure provides exemplary units and methods for the evaluation, identification, and production methods disclosed herein, a person of ordinary skill in the art will appreciate that performance of the evaluation, identification, and production methods herein is not limited to use of those units and/or methods. A person of skill in the art understands that although the use of assay conditions that differ in one or more aspects from those described herein for measuring a described parameter (e.g., amount of Th2 cytokines produced by a GA-specific Th2 cell when cultured in the presence of APCs and a test substance (e.g., GA) might give rise to different absolute values than those described herein, e.g., those described in Examples 2 and 3, a Th2 assay that uses different conditions or means (e.g., type of immunoassay or other assay) for measuring a described parameter (e.g., measuring intracellular cytokine levels rather than secreted levels in culture media) meets the disclosed predetermined range and/or value(s) even if the absolute range and/or value obtained by the Th2 assay are different, so long as the Th2 assay meets the herein disclosed predetermined range and/or value(s) when the herein disclosed assay conditions are used. Thus, by way of example, in certain examples provided herein, a ratio of APCs to Th2 cells of 3:1 is used to determine the predetermined range of GA-induced polypeptide levels (e.g., IL-4, IL-5, IL-6, IL-13, etc.). It is to be understood, however, that if other ratios of APCs to T cells are used, different (e.g., higher or lower) cytokine levels may be measured; however, such an assay is also encompassed by the present Th2 assay, since the same results would be obtained if the same ratios of APCs to T cells were used. The same would also be true for using different concentrations of antigen (e.g., GA), different culture times, different culture media, etc.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE FIGURES

FIGS. 1A and 1B contain bar graphs quantifying the levels (ng/ml) of the indicated cytokines (IL-2, IL12p40, IL-4, IL-5 (FIG. 1A), and IL-6, IL-10, IL-13 and IL-17 (FIG. 1B)) in the conditioned culture media of CD4+ T cells isolated from mice immunized with GA following two rounds of restimulation in the presence of antigen-presenting cells (APCs) and the indicated antigen or control (saline, ovalbumin (OVA), GA (Lot A) (“RLD”), or GA (“Lot B”). The bar indicates the mean and the error bars are the SEMs.

FIGS. 2A-2E contain graphical presentations of the representative response curves (cytokine levels (pg/ml) vs. concentration (μg/ml)) for the reference lot standard (“RLD”) of GA and glatiramoid (“COP-1” (Sigma-Aldrich)). Each data point indicates the mean and the error bars are the SEMs of triplicate measurements for IL-1β (FIG. 2A), KC and IL-4 (FIG. 2B), IL-2 and TNF-α (FIG. 2C), IL-5 and IL-12 (FIG. 2D), and IFN-γ and IL-10 (FIG. 2E).

FIG. 3 shows a graphical presentation of a representative IL-4 response curve (Normal Response (% Normalized optical density (OD)) vs. log GA concentration (μg/ml)) for the RLD (commercial preparation of GA) and a test preparation of GA (lot B). The response curve was fit to the four parameter logistic fit equation using nonlinear regression and the EC₅₀ (μg/ml) was calculated. One-way ANOVA followed by Tukey's multiple comparison test (significance level, p<0.05) was used for statistical testing of the dataset for each plate. There was no statistically significant difference between RLD and GA-induced IL-4 levels.

FIG. 4 is a line graph quantifying the concentration of IL-4 (pg/ml) detected in the GA-specific Th2 culture media, 24 hours after restimulation with the indicated antigens (RLD (commercial preparation of GA), MOG (35-55), PLP (139-151), MBP (87-89), MBP, OVA or COP-1) at each of the indicated concentrations of antigen.

FIG. 5 is a line graph quantifying the concentration of IL-4 (pg/ml) detected in the GA-specific Th2 culture media, 24 hours after restimulation with the indicated antigens (100% RLD (commercial preparation of GA), and non-conforming-GA containing a contaminating agent (CA) (i.e., 90% RLD+10% CA, 75% RLD+25% CA, or 25% RLD+75% CA)) at the indicated concentrations (Conc (μg/ml)). Each data point indicates the mean and the error bars are the SEMs of triplicate measurements.

FIG. 6 is a flow chart showing an exemplary protocol for generating GA-specific Th2 cells.

FIG. 7 is a flow chart showing an exemplary protocol for the Th2 copolymer assay. A test amino acid copolymer sample (e.g., GA) and 3 parts APCs are combined with 1 part GA-specific Th2 cells, generated, e.g., according to the method shown in FIG. 6, in FBS containing culture media for about 24 hours, at which time the supernatant is collected and the presence of one or more GA-induced polypeptides (e.g., one or more cytokines, such as, e.g., IL-4, IL-5, IL-6, IL-10, IL-13, etc.).

### DETAILED DESCRIPTION OF THE INVENTION

Other than molecular weight and amino acid composition, which are specified on the approved label for the product, the label and other available literature for Copaxone®, referred to herein as the reference drug lot (RLD), do not provide detailed information about the physiochemical characteristics of the product.

The present invention provides a highly reproducible, cell-based in vitro method for distinguishing glatiramer acetate (GA) preparations from certain non-conforming amino acid copolymers and/or to confirm that GA, or a sample, lot, or batch thereof, conforms to a reference value for GA. For example, in some instances, the present disclosure provides compositions related to polarized type 2 T helper (Th2) cells (e.g., GA-specific polarized Th2 cells) and methods of analyzing, selecting, and/or characterizing compositions related to co-polymers, e.g., antigens and GA. In some instances, methods herein include analysis of one or more polypeptide (e.g., cytokine) expression levels resulting from the polarized cells following exposure of the cells to a test antigen (e.g., a copolymer such as GA) and, in some methods, comparing the one or more levels to a reference standard (e.g., the level(s) resulting from a non-polarized T helper cell).

### Methods for Manufacture of Glatiramer Acetate

Generally, the process for the manufacture of glatiramer acetate includes three steps:

Step (1): polymerization of N-carboxy anhydrides of L-alanine, benzyl-protected L-glutamic acid, trifluoroacetic acid (TFA) protected L-lysine and L-tyrosine (collectively referred to as NCAs) to result in a protected copolymer,

Step (2): depolymerization and benzyl deprotection of the protected copolymer using hydrobromic acid in acetic acid, and

Step (3): deprotection of the TFA-protected lysines on the product copolymers followed by purification and drying of the isolated drug substance.

In Step (1) of the manufacturing method, the NCAs are co-polymerized in a predetermined ratio using diethylamine as an initiator. Upon consumption of the NCA components, the reaction mixture is quenched in water. The resulting protected polymer (Intermediate-1) is isolated and dried. In Step (2), the protected polymer (Intermediate-1) is treated with anhydrous 33% HBr in acetic acid (HBr/AcOH). This results in the cleavage of the benzyl protecting group on the glutamic acid as well as cleavage of peptide bonds throughout the polymer, resulting in a partially depolymerized product (Intermediate-2) with a reduced molecular weight relative to the parent Intermediate-1 polymer. After the reaction is quenched with cold water, the product polymer is isolated by filtration and washed with water. The Intermediate-2 material is dried before proceeding to Step (3). In Step (3), Intermediate-2 is treated with aqueous piperidine to remove the trifluoroacetyl group on the lysine. The resulting copolymer (Intermediate-3) is subsequently purified using diafiltration/ultrafiltration and the resulting acetate salt dried to produce Glatiramer Acetate drug substance.

Methods for the manufacture of glatiramer acetate have been described in the following publications: U.S. Pat. No. 3,849,550; WO 95/031990 and US 2007-0021324.

### GA-Specific Th2 Cells

The cells of the present disclosure have been generated to possess a phenotype useful in the assays disclosed herein. For example, the GA-specific Th2 cells described herein are relatively specific for GA, do not cross-react with certain other CNS proteins (e.g., one or more of MBP, MOG and PLP), express low levels of the proinflammatory Th1/Th17 cytokines (e.g., IFN-γ, IL-12 and IL-17), and express (e.g., and secrete) high levels (relative to that expressed in the absence of GA) of Th2 cytokines (e.g., one or more of IL-4, IL-5, IL-10, and IL-13) in the presence of GA, e.g., relative to non-polarized CD4+ T helper cells and/or GA-specific Th2 cells exposed to a non-conforming amino acid copolymer or protein (e.g., one or more of COP-1 and/or myelin basic protein (MBP)). In some instances the cells herein further express (e.g., and secrete) increased levels of one or more neurogenic and oligodendrogenic factors, such as, for example, brain derived neurotrophic factor (BDNF), neurotrophin 3 (NT3), GP-130 family members (e.g., leukemia inhibitory factor (LIF)), IL-6, IL-17, and/or GM-CSF, IL-2, IL-6, and/or TNF-α in the presence of GA and APCs, e.g., relative to non-polarized CD4+ T helper cells and/or GA-specific Th2 cells exposed to a non-conforming amino acid copolymer or protein (e.g., one or more of COP-1 and/or myelin basic protein (MBP)) in the presence of APCs.

The GA-specific Th2 cells described above can be generated as follows:

### Immunization

Optionally, a subject, such as a mouse (e.g., including, but not limited to, Balb/c, SJL/J, C57B1/6 mice, and other art-recognized strains), rat, or other mammal, or an in vitro cell population or line (e.g., an in vitro population of naïve spleen cells) is immunized or contacted with GA, e.g., to produce an antigen-specific immune response, e.g., against GA. Subjects are immunized at a suitable age, e.g., at about 9 weeks of age. For example, a mouse, such as a Balb/c mouse can be immunized with 250 μg GA, e.g., emulsified in a suitable adjuvant (e.g., CFA). After about 11 days (+/−10, 9 8, 7, 6, 5, 4, 3, 2, 1, or 0 days), the draining lymph nodes are isolated, a cell suspension is made, and CD4+ T cells are purified from the cell suspension. Longer time periods (e.g., up to one, two, three, six, or twelve months) can be used, e.g., if subjects are immunized with GA in incomplete frauds adjuvant (ICFA). In some instances, the lymph nodes from multiple immunized subjects, e.g., 2, 3, 4, 5, 6, 7, 8 or more, are obtained to increase the number of Th cells. The CD4+ T cells can be isolated according to any suitable method, e.g., using negative selection. In some instances negative selection, using, e.g., immunomagnetic beads, is used. The method for immunomagnetic bead purification of CD4+ T cells is well known in the art (see, e.g., Current Protocols in Immunology, Unit 3.5A, “Fractionation of T and B Cells Using Magnetic Beads”). In some instances, T cells can be obtained from a human subject exposed to GA or a GA-like drug product. Cells resulting from such methods can be used in the steps described below or can be stored in suitable conditions.

### Propagation: GA Stimulation/Re-stimulation

CD4+ T cell populations, optionally resulting directly from the above immunization, or from storage, can be incubated in suitable culture media (e.g., complete RPMI supplemented with a suitable concentration of fetal bovine serum (FBS), e.g., 1, 2, 3, 4, 5, 6, 7, 8, 9, 10%, or more FBS) in the presence of a population of antigen-presenting cells (APCs) and a concentration of GA. In some instances, APCs are either isolated or reanimated within about 24 hours of use. In some instances, the GA and the APCs are combined prior to adding the Th2 cells. The concentration of GA is typically about 20 μg/ml, although the concentration can range, e.g., from about 1 to 1000 μg/ml (e.g., 1-500, 1-250, 1-100, 1-50, 5-50, 10, 20, 30, 40, 50 μg/mL). In some instances, APCs, are treated with an “anti-proliferative agent,” which, as used herein, can include, e.g., mitomycin-C or irradiation, or any other suitable reagent or treatment that prevents cell proliferation. Any suitable APC can be used in the methods herein, including, for example, naïve, splenic APCs from a syngeneic subject. In some instances, APCs can be isolated e.g., from draining lymph nodes. Suitable APCs include, but are not limited to, for example, B cells, dendritic cells, microglia, astrocytes, and/or mixtures thereof. In some instances, APCs are treated with mitomycin-C. The ratio of APCs to CD4+ T cells is typically about 10:1, 5:1 or 3:1, although other ratios are possible (e.g., 9:1, 8:1, 7:1, 6:1, 4:1, 2:1, 1:1). Preferably the ratio is 10:1. The CD4+ T cells and APCs are incubated at 37° C., 5% CO₂, for about 2 days (e.g., about +/−1 day), or for a time suitable to maintain the phenotype of the cells. Cells resulting from the above can also be used in the assays disclosed herein and are referred to as “round 1” cells.

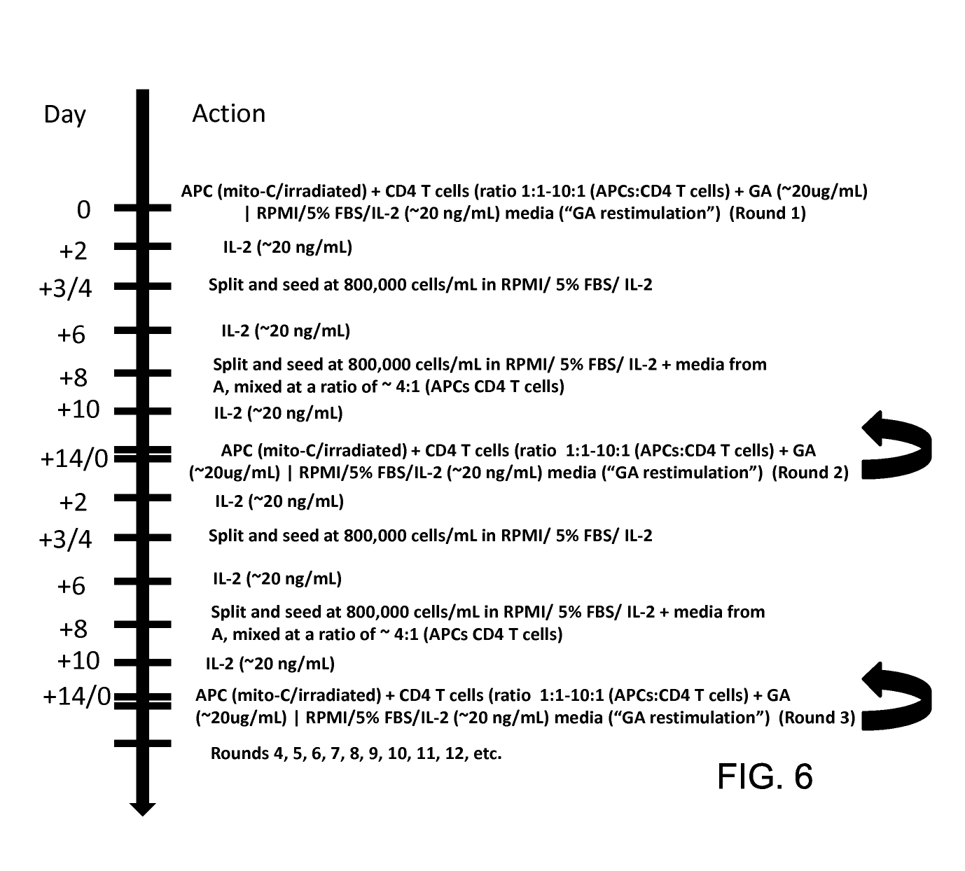

Following the 2 day incubation, the cells are resuspended in fresh media containing about 20 ng/ml IL-2. The cells are then cultured for an additional 2 days (e.g., about +/−1 day) or for a time suitable to maintain the phenotype of the cells, and the cells are split and reseeded at about 800,000 cells/mL in media containing 4 parts fresh media, 1 part media from the previous culture, and about 20 ng/ml IL-2. Excess cells are preserved under suitable conditions (e.g., at −80° C. in 95% FBS/5% DMSO). This cycle of adding IL-2-containing fresh media and reseeding the cells can be repeated for up to about 14 days (e.g., +/− about 1, 2, 3, 4, 5, 6, 7, or more days). At about 14 days (e.g., +/− about 1, 2, 3, 4, 5, 6, 7, or more days), cells can be restimulated with GA, as described above. Cells resulting from this restimulation are referred to as “round 2 cells”. Following restimulation, cells are propagated in IL-2 as described above until a next stimulation or until use or storage. Cells resulting from subsequent restimulations are referred to as round 3, 4, 5, 6 and so on. Any number of restimulations can be performed so long as the cells maintain the phenotype disclosed herein. Cells from any of these cycles can be used in the assays disclosed herein or stored as a cell bank, e.g., for use in the assays disclosed herein. In some instances, the cells can be, although not necessarily, restimulated as in the first round of stimulation (i.e., in the presence of GA and APCs), followed again by propagation; however, the GA-specific Th2 cells may also be used after the first round of stimulation and propagation, if desired. However, in a preferred embodiment, the cells are restimulated at least once. The cells may also be restimulated at least twice, at least three times, at least four times, five times, six times, seven times, eight times, or more. An exemplary protocol for generating the GA-specific Th2 cells of the invention is illustrated in the flow chart shown in FIG. 6.

As noted above, following the desired number of restimulations with GA and APCs, the GA-specific Th2 cells generally have the following properties or phenotype when exposed to GA and APCs: relatively high expression of IL-4, IL-5, IL-6, IL-10, and IL-13. When exposed to GA and APCs the cells also express (at a lower level than IL-4) IL-2 and TNF-α, and do not express (or express at only an extremely low level) MBP, MOG or PLP, and express only low levels of Th1/Th17 cytokines, IFN-γ, IL-12 and IL-17.

Preferably, the GA-specific Th2 cells of the invention express (e.g., and secrete, as measured, e.g., by levels in the culture media) 3-fold or greater, 4-fold or greater, 5-fold or greater, 6-fold or greater, 7-fold or greater, 8-fold or greater, 9-fold or greater, or 10-fold or greater levels of IL-4, IL-5, IL-16, IL-10 and/or IL-13, about 24 hours after restimulation with GA and APCs compared to the level of the cytokine(s) expressed in the presence of APCs and no GA, or in the presence of APCs and certain other polypeptides, peptides, or proteins (e.g., a non-conforming amino acid copolymer (e.g., one or more of COP-1 and/or MBP)). The GA-specific Th2 cells may then be used immediately in an assay, or banked (e.g., frozen, refrigerated, or otherwise appropriately stored) for future use. As long as the cells are frozen and thawed properly according to standard procedures, the cells maintain their viability and specific activity after being frozen and thawed (i.e., “reanimated”).

In a preferred embodiment, the GA-specific Th2 cells express more IL-4 when incubated in the presence of a given concentration of GA and APCs (e.g., naïve APCs) than when incubated in the presence of the same concentration of a non-conforming amino acid copolymer or protein (e.g., one or more of COP-1 and/or MBP) and APCs. Preferably, the Th2 cells express at least 2-fold or greater, at least 3-fold or greater, at least 4-fold or greater, at least 5-fold or greater, or at least 10-fold or greater (e.g., 1.000-fold or even 10.000-fold) IL-4 when incubated in the presence of a given concentration of GA and APCs than when incubated in the presence of the same concentration of the non-conforming amino acid copolymer or protein (e.g., one or more of COP-1 and/or MBP) and APCs. The preferred given concentration range of GA is from about 20 μg/ml to about 50 μg/ml.

Methods for the detection of polypeptides (e.g., IL-2, IL-4, IL-5, IL-6, etc., and MBP, PLP and MOG) are well known in the art. For example, polypeptide (e.g., cytokines) in the culture media can be detected using sandwich ELISA or multiplex assay (e.g., from Meso Scale Discovery). Intracellular and/or cell surface staining of Th2 cells may also be performed. Antibodies for detecting cytokines and polypeptides (e.g., MBP, MOG, PLP BDNF, LIF and NT3) are commercially available, e.g., from BD Bioscience (San Diego, Calif.), R&D Systems (Minneapolis, Minn.), Biolegend (San Diego, Calif.), and EBioscience (San Diego, Calif.). The level of mRNA encoding any of the above polypeptide can also be determined, e.g., by qPCR. The nucleic acid sequences of the above polypeptides are known to those of skill in the art, and the skilled artisan will know how to design primers for detecting the expression levels of a polypeptide according to well-known methods.

Methods of immunization for the in vivo generation of antigen-specific polyclonal CD4+ effector T cells (e.g., GA-specific T helper cells) are well known in the art (see, e.g., Aharoni et al. (1997) Proc. Natl. Acad. Sci. USA; 94:10821-10826; and Current Protocols in Immunology, “Production of Th1 and Th2 Cell Lines and Clones, Section 3.13.6, Supplement 72). Any suitable method for immunizing a mammal to generate a polyclonal GA-specific T cell response may be used to obtain GA-specific T cells for use as described herein.

### Th2 Assay

As stated above, the GA-specific Th2 cells described herein have the surprising and unexpected characteristic of being able to reproducibly distinguish GA from certain other non-conforming amino acid copolymers (e.g., ones which do not have the same ratio of L-glutamic acid, L-alanine, L-tyrosine, and L-lysine as GA, such as COP-1 (Sigma-Aldrich)) and/or to confirm that GA, or a sample, lot, or batch thereof, conforms to a reference value for GA.

Thus, a batch of a composition comprising an amino acid copolymer can be selected based on the response it induces when incubated with APCs and the GA-specific Th2 cells of the invention. To do so, a sample of the batch is incubated in the presence of the GA-specific Th2 cells and APCs (e.g., naïve APCs). Next the level of at least one GA-induced polypeptide expressed by the GA-specific Th2 cells at a predetermined time point is assayed (e.g., by ELISA or multiplex assay); and the batch is selected, as GA, if the level of the at least one GA-induced polypeptide is within a predetermined range. Pharmaceutical compositions can also be prepared according to the same method for selecting a batch, by combining at least a portion of the selected batch with a pharmaceutically acceptable carrier or diluent.

In a preferred embodiment, a batch of a composition comprising an amino acid copolymer (e.g., a composition comprising a candidate GA preparation) is selected and formulated as a drug product and/or identified as GA by incubating a portion of the batch (e.g., at a concentration of 50 μg/ml) in the presence of the GA-specific Th2 cells (prepared as described in Example 1, below) and naïve APCs at a ratio of 1:3 (Th2 cells to APCs), and then determining the level of at least one GA-induced polypeptide (preferably a Th2 cytokine such as IL-4, IL-5, IL-6, and/or IL-13) expressed by the GA-specific Th2 cells at a predetermined time point of 24 hours by ELISA (as described in Example 2, below), and then selecting the batch or identifying the composition in the batch as GA if the level of the at least one GA-induced polypeptide is within a predetermined range.

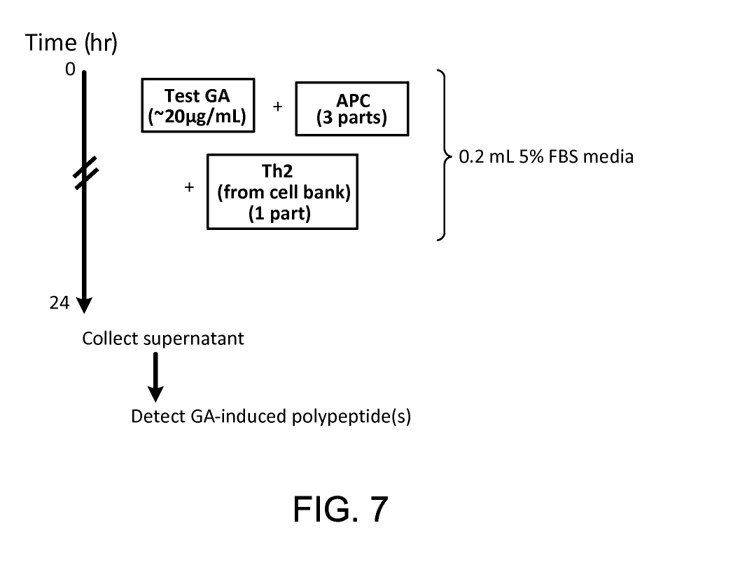

In another preferred embodiment, the batch of a composition comprising an amino acid copolymer (e.g., a composition comprising a candidate GA preparation) is selected and formulated as a drug product and/or identified as GA by incubating a portion of the batch (e.g., at a concentration of 50 μg/ml) in the presence of the GA-specific Th2 cells (prepared as described in Example 1, below) and naïve APCs at a ratio of 1:3 (Th2 cells to APCs), determining the level of IL-4, IL-5, IL-6, and/or IL-13 expressed by the GA-specific Th2 cells at a predetermined time point of 24 hours by ELISA (as described in Example 2, below), and selecting and formulating the batch or identifying the composition in the batch as GA if the level of IL-4, IL-5, IL-6, and/or IL-13 is within a predetermined range. The steps of the Th2 amino acid copolymer assay are exemplified in the flow chart shown in FIG. 7. In some instances, Th2 cells used in the assay can be round 2, 3, 4, 5, 6, 7, 8, 9, 10, 11, 12, or 13 cells. For example, cells used in the assay can be cells that were stored at round 12.

In certain embodiments, a “GA-induced polypeptide” is, e.g., IL-4, IL-5, IL-6, IL-10, IL-13, IL-2, or another polypeptide, e.g., disclosed herein, determined to be specifically induced upon culture with GA and APCs. In another embodiment, a “GA-induced polypeptide” is a Th2 cytokine (e.g., IL-4, IL-5, IL-6, IL-10, or IL-13). In yet another embodiment, a “GA-induced polypeptide” is selected from IL-4, IL-5, IL-10, and IL-13). Preferably, the conditioned culture media is assayed for secreted GA-induced polypeptides, although the expression level of one or more of the GA-induced polypeptides can also be determined, e.g., by intracellular or cellular staining and/or by qPCR. The culture media may be diluted for the assay.

The APCs and GA-specific Th2 cells can be combined, in the presence of an antigen (e.g., GA) in any ratio that results in expression of Th2 cytokines (e.g., IL-4), e.g., at levels detectable by ELISA and/or PCR. For example, the APCs and GA-specific Th2 cells are preferably incubated at a ratio of 10:1, more preferably 5:1 and most preferably 3:1. In some instances, the antigen (e.g., GA) and the APCs are combined prior to adding the Th2 cells. In some instances, an assay can include 150,000 APCs and 50,000 Th2 cells in 0.2 mL media. In some instances, an assay can include 75,000 APCs and 25,000 Th2 cells in 0.2 mL media.

The predetermined time point for which the cells are incubated before determining the expression of a GA-induced polypeptide is preferably about 24 hours, although longer or shorter incubation periods are possible. For example, a time point may be selected whereat expression of any one or more GA-induced polypeptides is optimal (e.g., detectable, e.g., by ELISA) and/or distinguishable from a level of expression for the same GA-induced polypeptide in the presence of a non-conforming copolymer (e.g., COP-1).

Suitable APCs include any cell that is capable of presenting GA-derived peptides in the context of major histocompatibility complex (MHC) class II molecules to the Th2 cells of the invention. Preferably, the APCs are naïve cells that do not express (e.g., secrete) GA-induced polypeptides (e.g., those disclosed herein). Suitable APCs include, for example, but not limited to, B cells, dendritic cells, microglia, astrocytes, and/or mixtures thereof. Suitable APCs can be obtained from the spleen, blood and/or lymph nodes of a naïve mammal. Preferably the APCs are syngeneic. In certain embodiments, a suitable APC is an immortalized cell line in which the cells express surface MHC class II molecules and are capable of presenting GA-derived peptides in the context of MHC class II molecules to the GA-specific Th2 cells.

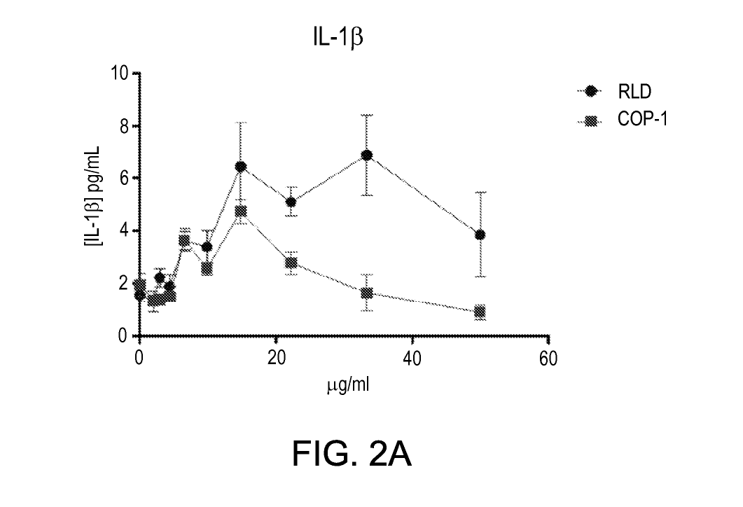

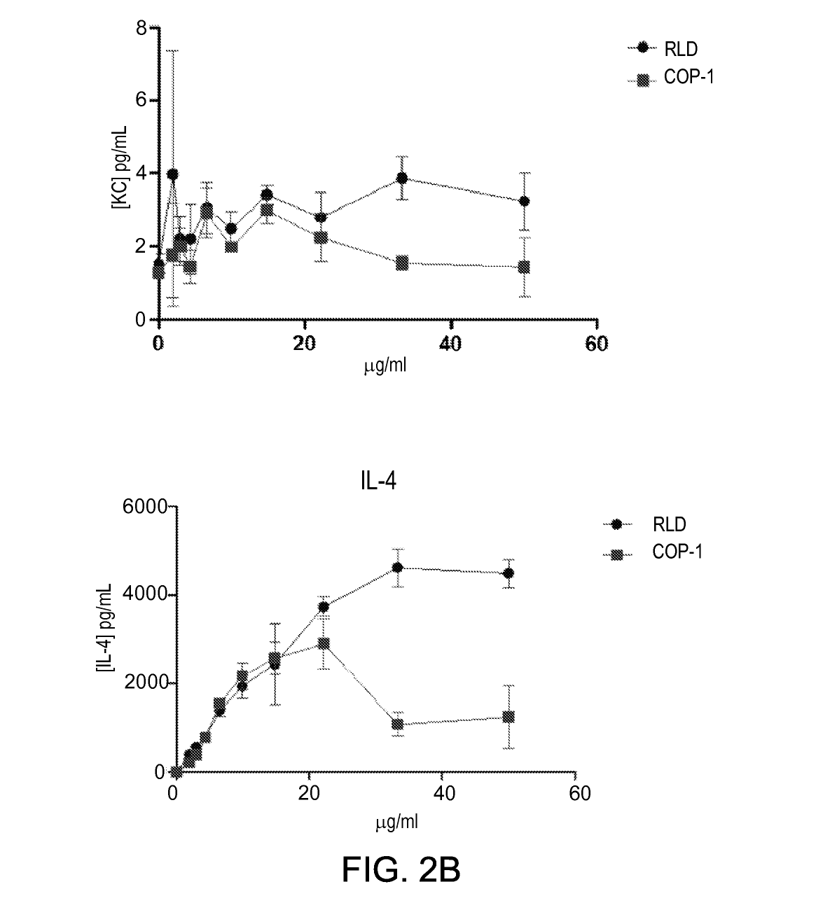

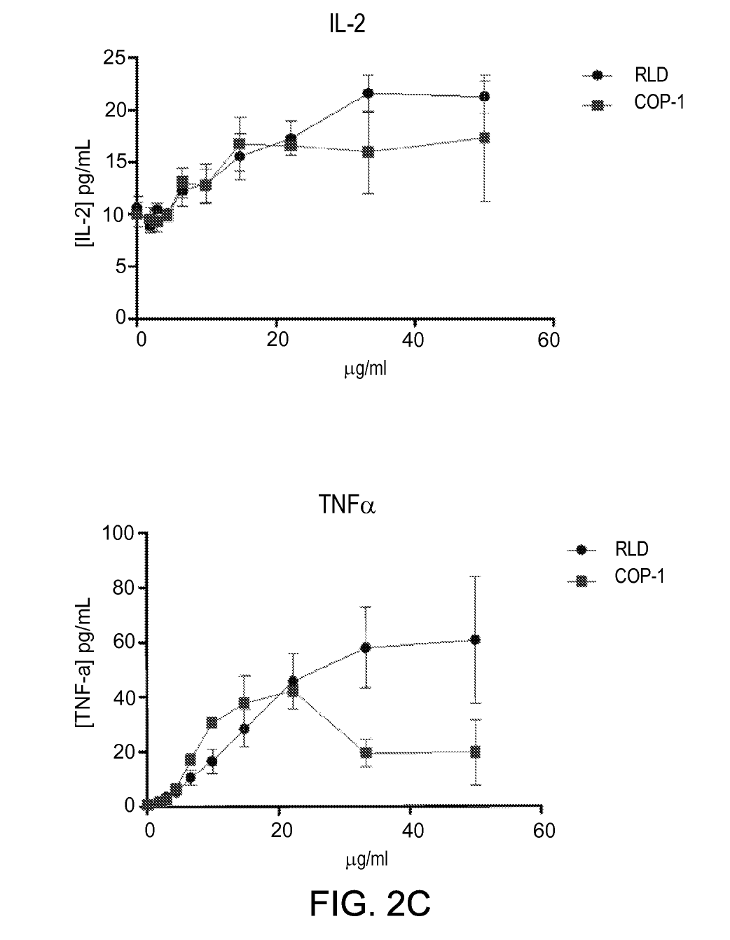

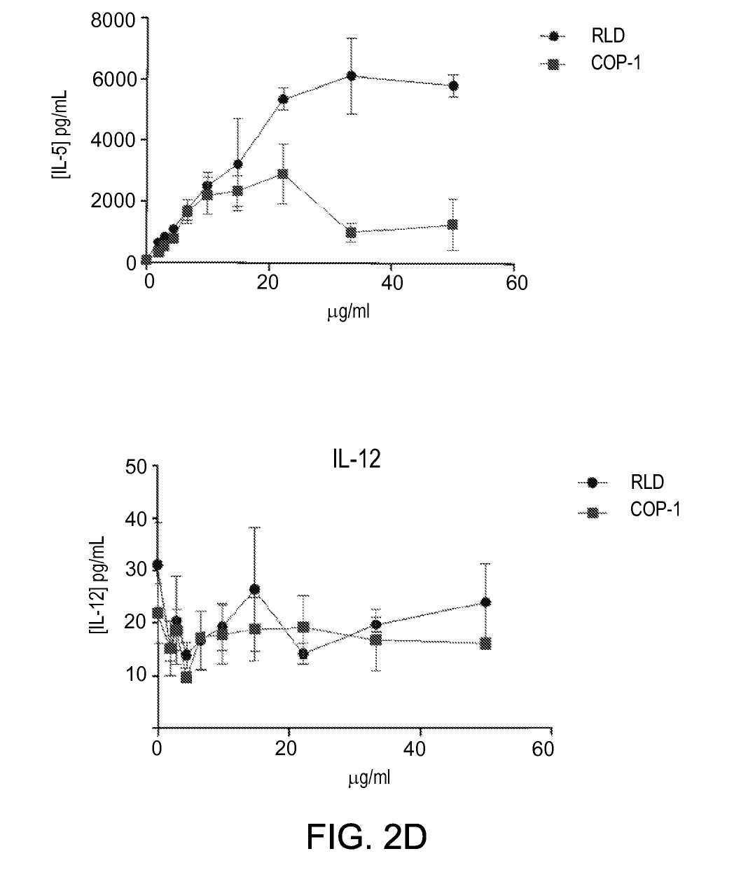

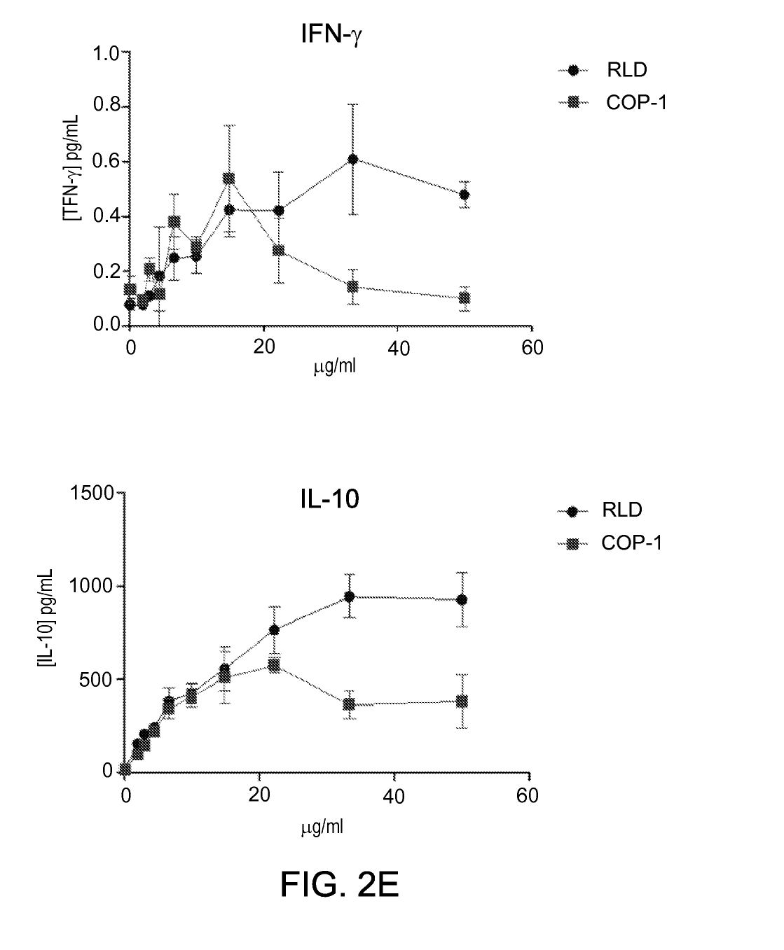

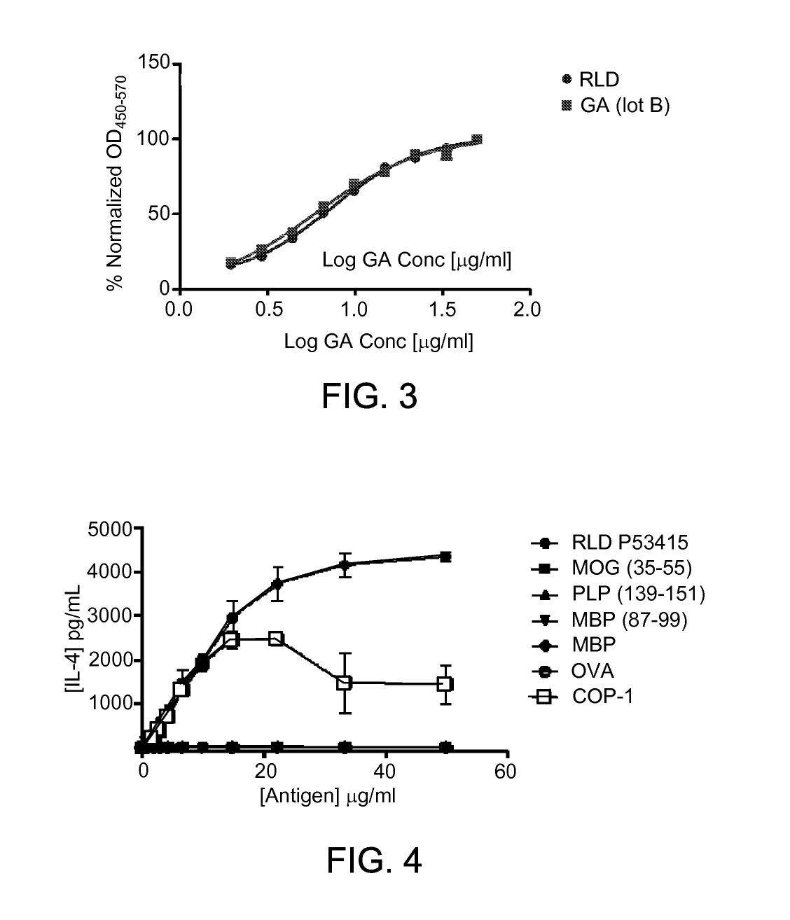

While the GA-specific Th2 assay of the invention can distinguish GA from certain non-conforming amino acid copolymers using a single concentration of GA, in certain embodiments, it may be preferable to test multiple amounts of the sample, in order to make a dose response curve, as exemplified in Example 2, FIGS. 2A-2E, and FIG. 3. Preferably, the concentration of copolymer in the sample tested in the assay ranges from about 10 μg/ml to about 50 μg/ml (0.1 μg/ml to 1000 μg/ml, e.g., 1 μg/ml to 100 μg/ml, 1 to 50 μg/ml, 10 to 50 μg/ml, 15 μg/ml, 20 μg/ml, 25 μg/ml, 30 μg/ml, 35 μg/ml, 40 μg/ml, or 45 μg/ml). When a single concentration of GA is used in the assay, the preferred amount is between about 30 μg/ml and 40 μg/ml.

In some instances, the predetermined range of the at least one GA-induced polypeptide may also be at least 1.5-fold, 2-fold, 3-fold, 5-fold or at least 10-fold increased compared to the level of the polypeptide expressed by the Th2 cells stimulated with APCs in the absence of GA, or in the presence of a different antigen, e.g., a non-conforming amino acid copolymer (e.g., one or more of COP-1 and/or MBP).

In some instances, the predetermined range for IL-4 is greater than 2 ng/ml, greater than 3 ng/ml, and preferably, greater than 4 ng/ml, when the T cells are restimulated with APCs and about 30 ng/ml GA. In another embodiment, the predetermined range for IL-5 is greater than 2 ng/ml, greater than 3 ng/ml, and preferably, greater than 4 ng/ml, greater than 5 ng/ml, or greater than 6 ng/ml, when the T cells are restimulated with APCs and about 30 ng/ml GA. In another embodiment, the predetermined range for IL-13 is greater than 1.5 ng/ml, when the T cells are restimulated with APCs and about 30 ng/ml GA. Preferably, in the above embodiments, the T cells are restimulated at about a 3:1 ratio APCs:T cells and the T cells are restimulated at a concentration of about 1×10⁶/ml, preferably in a final volume of about 200 μl.

### EXAMPLES

### Example 1

### Generation of GA-Specific Polarized Th2 Cells

This example describes the generation of a population of GA-specific type 2 T helper (Th2) cells that are capable of secreting high levels of Th2 cytokines as well as low levels of IL-2 and TNF-alpha, in response to restimulation with APCs and GA or the RLD (commercial preparation of GA(Copaxone®)).

Balb/c mice (Jackson Labs) were immunized with 250 μg/mL of GA (reference lot drug (RLD) or ovalbumin (OVA), each in saline, emulsified in complete Freund's adjuvant (CFA) on day 1. On day 11, the draining lymph nodes were harvested, single cell suspensions were made, and CD4+ T cells were enriched by negative selection immunomagnetic bead purification.

For ex vivo challenge, CD4+ T cells (1×10⁶/ml) were re-stimulated with 20 μg/mL of the same antigen used to immunize the mice (RLD, GA, or OVA) in the presence of freshly isolated or frozen naïve mitomycin C-treated splenic APCs (1×10⁷/ml) for 3-4 days, at which time, the culture media was replaced with fresh culture media supplemented with 20 ng/ml of IL-2. Cells were split and reseeded at 800,000 cells/mL in media containing 4 parts fresh media (RPMI/5% FBS/20 ng/mL IL-2) and 1 part recycled media from the previous culture. This cycle of changing the media and splitting the cells was repeated for 6 cycles. Cells were frozen and stored as a cell bank.

After cell propagation, the CD4+ T cells were restimulated once again with freshly isolated naïve mitomycin C-treated splenic APCs (1×10⁷/ml) for 4 days and 20 μg of the respective antigen. Culture supernatants were collected 72 hours after the start of each restimulation for analysis. The method for generating the GA-specific Th2 cells is summarized in the flow chart shown in FIG. 6.

Th1 (IL-2 and IL-12p40), Th2 (IL-4, IL-5 IL-6, IL-10, and IL-13) and Th17 (IL-17) cytokine levels (ng/ml) were measured using multiplex technology (Endogen Searchlight, Rockford, Ill.). A one-way ANOVA followed by Newman-Keuls multiple comparison test (significance level, p<0.05) for each individual measured cytokine was used for statistical analysis.

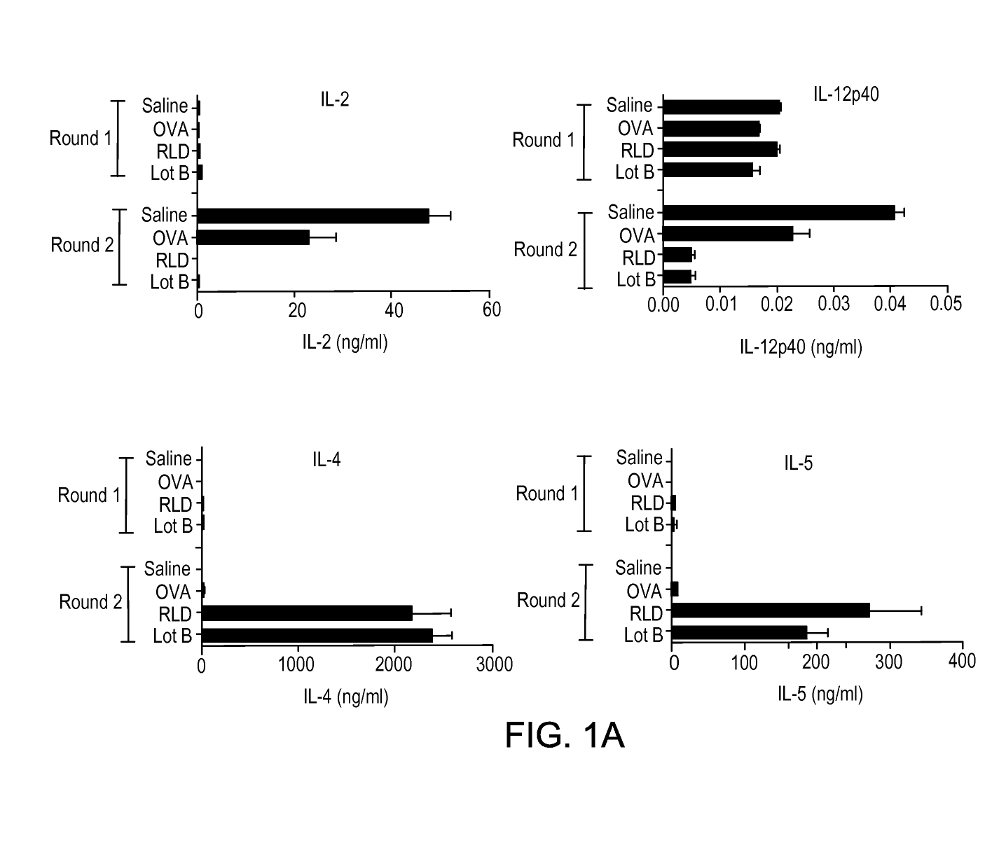

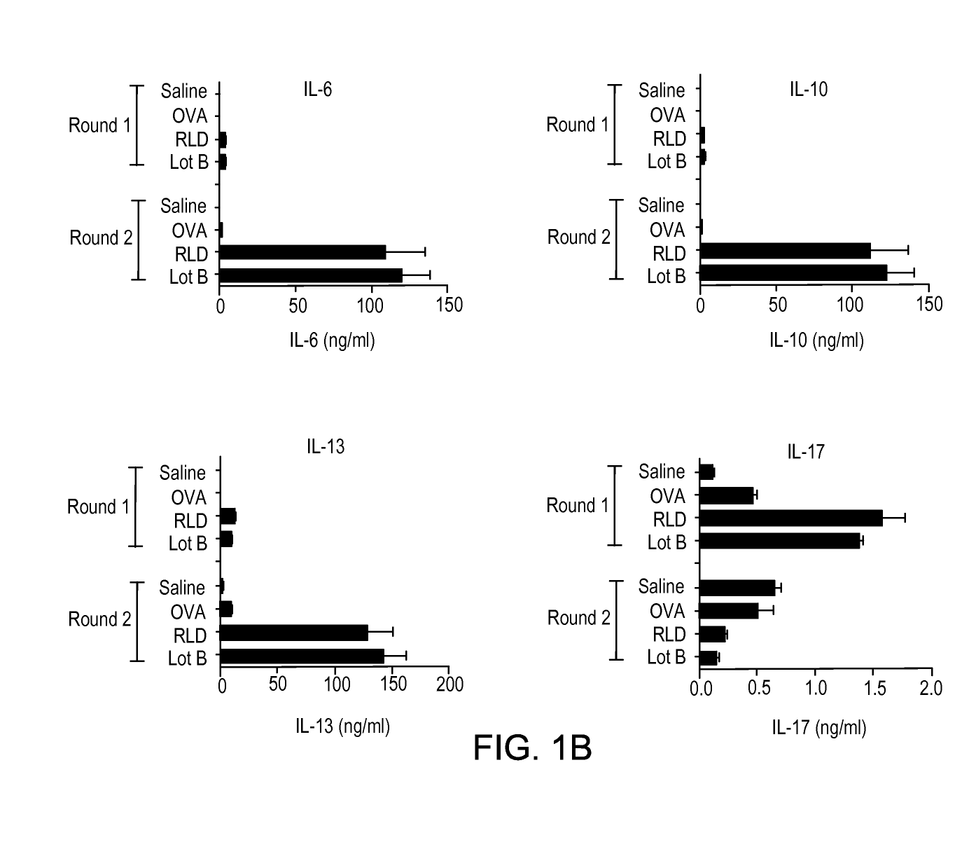

As shown in FIG. 1A and FIG. 1B, CD4+ T cells were polarized toward a Th2 phenotype following two rounds of stimulation with GA. After the first round of stimulation, GA induced low but detectable levels of most Th2 cytokines, such as IL-5, IL-6, IL-10, and IL-13. After a second round of stimulation, high levels of Th2 cytokines were selectively induced by GA. In contrast, the pro-inflammatory Th17 cytokine, IL-17, and the Th1 cytokine (IL-12p40) were selectively down-regulated with the second round of stimulation. T cells stimulated with ovalbumin did not show a corresponding increase in Th2 cytokines.

These results demonstrate that GA-specific Th2 cells generated according to this protocol secrete high levels of Th2 cytokines in response to stimulation with GA and APCs.

### Example 2

### Th2 Assay for Distinguishing GA from Non-conforming Amino Acid Copolymers

This example demonstrates that the GA-Th2 cells described in Example 1 can be used in an assay to distinguish GA from certain non-conforming amino acid copolymers (e.g., those that have different ratios of L-glutamic acid, L-alanine, L-tyrosine, and L-lysine), such as COP-1.

Populations of Th2 cells and APC cells were reanimated in culture media (RPMI/5% FBS/20 ng/mL IL-2), added dropwise. Cells were pelleted at 400-800G before being resuspended in fresh culture media for use in the assay. For the assay, 150,000 APC cells were combined with a concentration of GA using serial dilution of the GA. The resulting concentration curve provided a range from 0-50 μg/mL GA. APC cells were incubated with the GA for about 10 minutes before the Th2 cells were added. The final volume for the assay was 0.2 mL. The set up for the Th2 assay is illustrated in FIG. 7.

More specifically, GA-specific polarized Th2 cells prepared according to Example 1 were cultured in vitro with varying concentrations of GA (reference standard commercial preparation, Copaxone® or glatiramoid (Sigma-Aldrich (COP-1)) in the presence of syngeneic, mitomycin-C-treated, naïve splenic APCs. Cells were cultured in 96-well plates in a final volume of 200 μl per well, with 50 μl of irradiated splenic APCs (150×10³/50 μl) freshly isolated from naïve Balb/c mice, 50 μl of GA-specific polarized Th2 cells (50×10³/50 μl) and 100 μl of culture media (complete RPMI-10 supplemented with 10% FBS) containing RLD or COP-1 at increasing concentrations of GA from 0 to 50 μg/ml. Conditioned culture media was collected 24 hours post stimulation for analysis by multiplex assay and ELISA. Th1 (IL-113, IL-2, IL-12p70, TNF-α, KC and IFN-γ) and Th2 (IL-4, IL-5, and IL-10) cytokine levels (pg/ml) were measured using the 9-plex mouse Th1/Th2 cytokine kit from Meso Scale Discovery (MSD) (Gaithersburg, Md.).

The cytokine levels measured in the culture media following restimulation are shown in FIGS. 2A-2E. Both RLD and COP-1 showed a dose-dependent increase of high levels (μg/ml) of the Th2 cytokines (IL-4, IL-5, and IL-10) (FIGS. 2B, 2D and 2E) in comparison to the Th1 cytokines The response of COP-1 was readily distinguishable from RLD in terms of the secretion of the Th2 cytokines The levels of the secreted Th1 cytokines (IL-1β, KC, and IFN-γ) (FIGS. 2A, 2B and 2E) were very low (below limits of quantitation) and not GA dose dependent. The levels of IL-12 could be quantitated but the secretion of this cytokine was not dose dependent. Levels of IL-2 and TNF-α were quantifiable and exhibited dose dependency (FIG. 2C). COP-1 was discriminated from RLD at the higher doses (>˜30 μg/ml) in terms of levels of IL-2 and TNF-α secreted (FIG. 2C).

### Example 3

### Comparison of Glatiramer Acetate to Reference Lot Drug (RLD)

This example demonstrates that GA and RLD (Copaxone®) have similar IL-4 profiles as determined using the GA-specific polarized Th2 assay described in Example 2, above.

GA-specific polarized Th2 cells prepared as described in Example 1 were incubated in the presence of freshly isolated irradiated Balb/c APCs and RLD or GA (lot B) at the concentrations ranging from 0-50 μg/ml. The assay was run as described in Example 2, above, and conditioned culture media were collected after 24 hours for quantitation of IL-4 levels by ELISA.

FIG. 3 shows a graphical presentation of the representative IL-4 response curves for the RLD and GA. The response curve was fit to the four parameter logistic fit equation using nonlinear regression and the EC₅₀ (μg/ml) was calculated. One-way ANOVA followed by Tukey's multiple comparison test (significance level, p<0.05) was used for statistical testing of the dataset for each plate.

There was no statistically significant difference observed between the RLD and GA. Thus, it was concluded that the sample of GA was equivalent to the RLD in the expression of IL-4 by the GA-specific Th2 cells.

### Example 4

### Antigen-Specificity of the GA-Specific Polarized Th2 Cells

This example demonstrates that the GA-Th2 cells described in Example 1 are highly specific for GA, and do not cross-react with encephalitogenic peptide based brain neuroantigens.

The GA-Th2 assay was carried out as described in Example 2, except that the cells were restimulated with freshly isolated irradiated Balb/c APCs and RLD, MOG (35-55) (Peptides International, Louisville, Ky.), PLP (139-151) (Peptides International), MBP (87-89) (Peptides International Inc., Catalog No. PMB-3973-PI, lot #918342), murine MBP (EMD Biosciences, San Diego, Calif., Catalog No. 124027, Lot No. #D00095389), OVA (Endograde, from Profos, Germany) or COP-1 (Sigma-Aldrich) at concentrations from 0 to 50 μg/ml, as indicated in FIG. 4. IL-4 levels were determined by sandwich ELISA. As shown in FIG. 4, the polarized Th2 cells did not cross react with other CNS proteins, such as MBP, MOG, and PLP, since no IL-4 was detected in the culture media in those groups.

### Example 5

### GA-Specific Polarized Th2 Cells Distinguish GA from GA Preparations Containing Non-GA Material

This example demonstrates that GA manufactured to include defined levels of contaminating agent (i.e., a non-conforming-test-GA) could be distinguished from a composition containing conventionally manufactured GA, using the GA-Th2 assay described in Example 2.

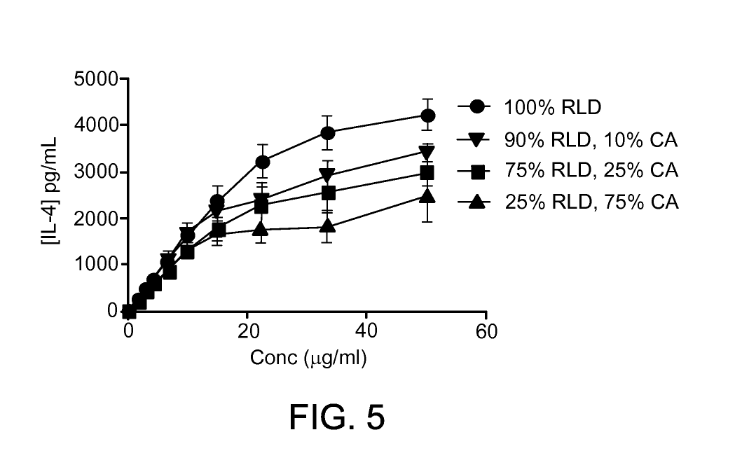

The GA-Th2 assay was carried out as described in Example 2, except that the cells were restimulated with 100% RLD (commercial preparation of GA), 90% RLD+10% contaminating agent, 75% RLD+25% contaminating agent, or 25% RLD+75% contaminating agent. As shown in FIG. 5, the assay could distinguish all preparations of non-conforming-test-GA from the 100% RLD preparation, at concentrations ranging from 20 to 50 μg/ml.

These results demonstrate that the GA-specific Th2 assay is highly sensitive and can be used to identify and distinguish a conforming and a non-conforming preparations of GA. These results also show that the assays disclosed herein can be used to detect GA resulting from altered manufacture and/or contaminated GA.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
incubating at least one sample of a batch of a composition comprising glatiramer acetate (GA) in the presence of antigen-presenting cells (APCs) and a population of cells comprising polyclonal GA-specific type 2 T helper (Th2) cells, wherein the population of cells has less than 1% cross-reactivity with myelin basic protein (MBP);
determining the level of at least one GA-induced polypeptide expressed by the population of cells at a predetermined time point; and
formulating at least a portion of the batch as a drug product if the level of the at least one GA-induced polypeptide is within a predetermined range.

2. The method of claim 1, wherein the population of cells has a cross-reactivity with MBP of less than 0.5%.

3. The method of claim 1, wherein the population of cells, when incubated for 24 hours at a concentration of 1×10⁶ cells/ml in the presence of APCs and at least 15 μg/ml GA, produce at least 100-fold more interleukin (IL)-4 than when incubated in the presence of APCs and MBP.

4. (canceled)

5. The method of claim 1, wherein the predetermined range is 125% to 75% of the level of the same GA-induced polypeptide expressed by the population of cells when incubated under the same conditions with a reference sample.

6. The method of claim 5, wherein the reference sample is a sample of commercially available pharmaceutical preparation of GA.

7. The method of claim 1, wherein the population of cells expresses about 1.5 fold more IL 4 when incubated for at least 24 hours in the presence of at least 25 μg/ml GA and APCs than when incubated in the presence of the same concentration of poly(Ala, Glu, Lys, Tyr) 6:2:5:1 hydrobromide having a molecular weight of 20,000-30,000 Daltons and APCs.

8. The method of claim 1, wherein the at least one GA-induced polypeptide is IL-4.

9. The method of claim 8, wherein the predetermined range of IL-4 is 3000-5000 pg/ml when the population of cells is incubated at a concentration of 1×10⁶ cells/ml for at least about 24 hours in the presence of the APCs and at least about 25 μg/ml of the sample.

10. The method of claim 1, wherein the population of cells comprises a population of isolated polyclonal GA-specific Th2 cells.

11. The method of claim 1, wherein the population of cells is non-human.

12. The method of claim 1, wherein the population of cells is murine.

13. (canceled)

14. The method of claim 1, wherein the predetermined time point is at least 24 or 48 hours after starting the incubation of the composition comprising GA in the presence of the population of cells and the APCs.

15. The method of claim 14, wherein the population of cells is incubated with the APCs at a ratio in the range of 1:3 to 1:10 (population of cells:APCs).

16. The method of claim 15, wherein the population of cells is incubated with the APCs at a ratio of 1:3 (cells:APCs).

17. The method of claim 1, wherein the formulating step comprises adding mannitol to the portion of the batch.

18. The method of claim 1, wherein the batch of a composition comprising GA is a drug substance.

19. The method of claim 1, wherein the batch of the composition comprising GA is prepared by a method comprising:
polymerizing N-carboxy anhydrides of L-alanine, benzyl-protected L-glutamic acid, trifluoroacetic acid (TFA)-protected L-lysine, and L-tyrosine to generate a protected copolymer (Intermediate-1);
treating the protected copolymer to partially depolymerize the protected copolymer and to deprotect benzyl protected groups thereby producing Intermediate-2;
treating Intermediate-2 to deprotect TFA-protected lysines thereby producing Intermediate-3; and
processing Intermediate-3 to produce GA.

20. A method of identifying a batch of a composition comprising a copolymer of tyrosine, lysine, alanine and glutamic acid as a batch of a composition comprising GA, the method comprising:
providing a batch of a composition comprising a copolymer of tyrosine, lysine, alanine and glutamic acid;
incubating at least one sample of the batch in the presence of APCs and a population of cells comprising polyclonal GA-specific Th2 cells, wherein the population of cells has less than 1% cross-reactivity with MBP;
determining the level of at least one GA-induced polypeptide expressed by the population of cells at a predetermined time point; and
identifying the batch of the copolymer as GA if the level of the at least one GA-induced polypeptide is within a predetermined range.

21. A method comprising:
incubating at least one sample of a batch of a composition comprising GA in the presence of APCs and a population of cells comprising polyclonal GA-specific Th2 cells, wherein the population of cells has less than 1% cross-reactivity with MBP and, when incubated for 24 hours at a concentration of 1×10⁶ cells/ml in the presence of APCs and at least 15 μg/ml GA, produces at least 100-fold more IL-4 than when incubated in the presence of APCs and MBP;
determining the level of at least one GA-induced polypeptide expressed by the population of cells at a predetermined time point; and
formulating at least a portion of the batch as a drug product if the level of the at least one GA-induced polypeptide is within a predetermined range.

22. The method of claim 21, wherein the predetermined time point is at least 24 or 48 hours after starting the incubation of the composition comprising GA in the presence of the population of cells and the APCs.
